(19) United States
(12) Patent Application Publication
(10) Pub. No.: US 2008/0299186 A1
(43) Pub. Date: Dec. 4, 2008

(54) COATINGS FOR APPLYING SUBSTANCES ONTO SUBSTRATE CARRIER

(75) Inventors: Glenn E. Fritz, Arlington, TN (US); Joseph P. Reo, Lakeland, TN (US); Mohammed A. Kabir, Lakeland, TN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering-Plough Healthcare Products, Inc.

(21) Appl. No.: 12/130,225

(22) Filed: May 30, 2008

Related U.S. Application Data
(60) Provisional application No. 60/941,577, filed on Jun. 1, 2007.

(51) Int. Cl. A61K 9/48; A61K 31/137; A61K 9/20; A61K 47/10

(52) U.S. Cl. 424/451; 514/653; 514/772; 424/464

(57) ABSTRACT

Compositions of matter containing an active agent, and methods of manufacturing thereof, wherein the method comprises the step of coating a substrate with a coating composition comprising the active agent formulated for immediate release, wherein the coating composition does not contain an appreciable amount of cellulosic materials and preferably comprises polyvinyl alcohol or a polyvinyl alcohol derived copolymer.

### BACKGROUND

Identification or discussion of any reference in this section or any part of this specification shall not be construed as an admission that such reference is available as prior art to the present application. All references cited herein are hereby incorporated in their entirety into the subject application.

The subject invention relates to formulations containing active substances applied to substrates for consumption by a subject in order to gain immediate release of the active substance in the subject. Such systems are known in the art of pharmaceutical manufacturing where active substances have been applied to inert substrates or tablet substrates containing additional therapeutic agents. The substrates themselves can be formulated for immediate release or delayed release of such additional therapeutic agents. In particular, the subject invention provides coating systems containing polyvinyl alcohol (PVA), including PVA derived copolymers, as coating excipients for active substances containing primary or secondary amine moieties. More particularly the invention relates to the use of coating systems that do not contain cellulosic materials as coating excipients for active substances containing primary or secondary amine moieties.

One of the obstacles to be overcome in manufacturing of pharmaceutical and similar formulations is the prevention of degradation impurities of active ingredients over a products' shelf life, which could exceed 2 years. For example, international pharmaceutical manufacturing guidelines require producers to identify any individual degradation product that exists at a level of at least 0.2% (w/w) of the initial drug product and safety studies are required for any degradation product that exists at a level of at least 0.5% (w/w) of the initial drug product. See, e.g., International Conference of Harmonization (ICH) of Technical Requirements for Registration of Pharmaceuticals for Human Use Q3B(R)″ (Impurities in New Drug Products, revised 5 Feb. 2003). As a result, producers continually monitor drug formulations for the presence of degradation products in stability studies. Interactions between active ingredients and formulation aids have typically been obviated by the use of protective barriers or subcoatings around active ingredients. See, e.g., U.S. Pat. No. 6,607,747. However, barriers and subcoatings are not feasible solutions for degradation products resulting from the interaction of the active ingredients and film forming agents in immediate release coatings.

Cellulosic polymers such as hydroxypropyl methylcellulose (HPMC) have been a preferred polymer for applying drugs to tablet surfaces due to unique physical properties. For example, HPMC, which is known to have a high tensile strength depending on the grade or molecular weight used, is the film former most often chosen for immediate release coating compositions. Because of the high tensile strength, the addition of many water soluble compounds, including pharmaceutical compounds, can act as plasticizing agents to aid in adhesion and elongation of HPMC-based film. HPMC also has a relatively high viscosity in water which makes it very suitable for acting as a viscosity modifier agent to suspend non aqueous soluble drugs in the dispersion. However, as explained herein, it has been unexpectedly found that for compositions containing active ingredients containing primary or secondary amine moieties in immediate release coatings, the active ingredients were found to produce higher levels of chemical degradation and formation of impurities when incorporated into coating compositions containing cellulosic polymers, e.g. hydroxypropyl methylcellulose and hydroxypropylcellulose.

Polyvinyl alcohol (PVA) is used as a binder in such things as glues and cement mixes and has been used as a binder in tablet formulations. When used as a tableting aid, PVA is typically applied by spraying and it maintains a high degree of tackiness. PVA has also been used for aesthetic film coating applications, but requires addition of significant amounts of detackifiers and other excipients to obtain a functional coating for a pharmaceutical dosage form. Modification of those formulated systems containing PVA are not recommended by their manufacturers. PVA is also known to aid in adhesion and elongation. Thus, adding materials with similar properties to PVA (such as noted above for active ingredients added to cellulosic materials) would be expected to negatively impact the final coating dispersion formulation due to excessive tackiness and excessive adhesion. Further, because PVA does not maintain high levels of viscosity, suspending insoluble drugs requires extensive process evaluation and optimization. However, it has been unexpectedly found that the addition of water soluble and insoluble active pharmaceutical ingredients, did not significantly affect the film formation properties of the final product containing PVA.

Thus, it would be beneficial to have an immediate release coating formulation for applying an active ingredient containing at least one primary or secondary amine moiety to a substrate that preserves the stability of the active substance. Further, it would be beneficial to have a composition that readily provides a film for applying an immediate release of active ingredients to tablet formulations. These and other advantages of the subject invention are described more fully below.

### SUMMARY OF THE INVENTION

The subject invention provides a method of manufacturing a composition of matter for consumption by an animal, wherein the composition of matter comprises a compound containing a primary or secondary amine moiety, the method comprising the step of coating a consumable substrate with a coating composition comprising said compound, wherein said coating composition does not contain an appreciable amount of cellulosic materials.

The subject invention further provides a pharmaceutical composition comprising a pharmaceutically active agent seated on a substrate, wherein the pharmaceutically active agent comprises a compound containing a primary or secondary amine moiety and the pharmaceutical composition does not contain an appreciable amount of cellulosic material.

The subject invention also provides a pharmaceutical composition comprising a pharmaceutically active agent seated on a substrate, wherein the pharmaceutically active agent comprises a compound containing a primary or secondary amine moiety and wherein the pharmaceutically active agent is applied to the substrate as part of a composition that does not contain an appreciable amount of cellulosic materials.

The subject invention further provides a pharmaceutical composition comprising an immediate release coating composition on a substrate, the coating comprising a therapeutically active agent containing at least one primary or secondary amine moiety, the immediate release coating consisting essentially of the therapeutically active agent and polyvinyl alcohol or polyvinyl alcohol derived co-polymer.

The subject invention also provides a pharmaceutical composition comprising an immediate release coating composition on a substrate, the coating comprising a therapeutically active agent containing at least one primary or secondary amine moiety, the immediate release coating consisting of the therapeutically active agent and polyvinyl alcohol or polyvinyl alcohol derived co-polymer.

The subject invention further provides a method of manufacturing a composition of matter comprising applying to a substrate a coating composition comprising an active ingredient and a film forming agent, wherein the film forming agent comprises a polyvinyl alcohol or a polyvinyl alcohol derived copolymer, wherein the coating composition provides for immediate release of the active ingredient.

### DETAILED DESCRIPTION OF THE INVENTION

The compositions produced according to the methods described herein are intended for consumption by an animal, including human and non-human animals.

In one embodiment of the invention, the active pharmaceutical agent containing a primary or secondary amine is phenylephrine or a pharmaceutically acceptable salt thereof, which can, for example, be formulated into a coating dispersion and applied as a film to a consumable substrate.

In one embodiment of the invention, the coating composition comprises polyvinyl alcohol (PVA) or polyvinyl alcohol derived copolymer. In this description PVA and PVA derived copolymers may both be referred to as PVA polymers. As used herein, polyvinyl alcohol derived copolymer refers to a copolymer that contains at least one PVA monomer unit. The PVA or PVA derived copolymer may comprise PVA of any molecular weight in the final polymer composition. In one embodiment of this aspect of the invention, the coating composition contains PVA polymer as a non-ionic, water soluble, film forming polymer/co-polymer formulation system. Other components of this coating composition can comprise additional formulating aids such as polyalkylene glycols, such as polyethylene glycol (PEG) of any grade or molecular weight, and additional processing aids such as talc and titanium dioxide, polysorbate 80 and others known to be used in such commercially available systems. In additional embodiments of the invention, PVA polymer can be synthesized as a portion of the copolymer composition such as a PVA/polyalkylene glycol of any molecular weight in a graft co-polymer. PVA polymer in amounts between 3 and 180 mg/dose, preferably between 10 and 50 mg/dose, most preferably between 18 and 36 mg/dose. In certain example embodiments of the invention, the coating compositions comprise a film forming agent consisting essentially of PVA or PVA derived copolymer. In certain addition example embodiments, the coating compositions comprise a film forming agent consisting of PVA or PVA derived copolymer.

As used herein the term no appreciable amount of cellulosic materials means no amount of cellulosic material in the compositions that would contact with the active ingredient containing a primary or secondary amine moiety and promote or enhance degradation of the active ingredient. In certain embodiments of the invention, the coating compositions are substantially free of cellulosic materials. In certain additional embodiments, the coating compositions are free of cellulosic materials to the extent detectable by routine analytical methods. Numerous examples of cellulosic materials are known to those in the art of formulating pharmaceutical formulations or formulations for consumption by humans and other animals. Cellulosic materials are included, for example, as fillers, gellants, and matrix materials. As used herein cellulosic materials include, but are not limited to, polysaccharides such as hydroxypropylmethylcellulose (HPMC), hydroxypropylcellulose (HPC) hydroxyethylcellulose (HEC), carboxymethylcellulose (CMC), hydroxyethylcellulose (HEC), methylcellulose (MC), ethylhydroxyethylcellulose (EHEC), hydroxyethylmethylcellulose (HEMC), hydrophobically modified hydroxyethylcellulose (HMHEC), hydrophobically modified ethylhydroxyethylcellulose (HMEHEC), carboxymethylhydroxyethylcellulose (CMHEC), and carboxymethyl hydrophobically modified hydroxyethylcellulose (CMHMHEC).

In the practice of this invention the PVA polymer coating formulation may optionally comprise other co-processing aids including but not limited to additional polymers, including other grades or blends of PVA polymer, colorants, opacifiers, plasticizers, surfactants, anti-foam agents, emsulsifiers, viscosity modifying agents and detackifiers. Each of these may be present in amounts up to 75% w/w, preferably in amounts up to about 30% w/w.

In a preferred embodiment, the PVA polymer coating formulation consists of only PVA of any molecular weight or PVA contained in a copolymer, for example, Kollicoat® IR (BASF, N.J. USA) or as part of a PVA based coating system such as the various film coating products available under the trade name Opadry® (Colorcon, PA, USA), for example Opadry II® 85F series, Opadry® II 85G series or Opadry® AMB®, and one or more pharmaceutically active compounds.

In a preferred embodiment where the active ingredient is phenylephrine, the pH of the dispersions should be maintained between pH 2-7 and preferably between pH 3-6 until processed or sprayed so as to maintain optimal chemical stability of the phenylephrine. Those of ordinary skill in the art may determine that similar manipulation of pH may be useful for additional active ingredients useful in the claimed invention.

In the practice of the invention these dispersions are formulated in any solvent system, but preferably in a predominantly aqueous system.

In one embodiment of the invention the coating formulation is formed as a dispersion. In a separate embodiment, the coating formulation can be formed as a solution. In either form, the coating formulation can be applied as a coating on a consumable substrate, such as a tablet surface, granule surface, or can be co-processed with any inert powder or substrate with the intention of providing immediate release pharmaceutical agent. These coated particles or granules can then be used on their own as a raw material or compressed or incorporated into any other final immediate release dosage form. In addition, the coated substrate can be incorporated into additional materials that provide a delayed release of the materials, such as a caplet or capsule.

As used herein, the term applying the coating formulation is intended to cover all means of applying a coating to a substrate, including, spray coating, roll coating, spinning disc coating among others. As used herein, the term seated on a substrate means the presence of the coating formulation on the substrate such as would result from the applying step. In certain embodiments of the invention the coating formulation is spray coated on a substrate.

As used herein, the term immediate release is used as it is known in the art of pharmaceutical formulation and is intended to cover all such formulations providing for release of at least 50% of the active ingredient within 5 to 60 minutes of contact with a physiological system. In typical immediate release formulations the majority of the active ingredient is released within two hours.

The term active ingredient, as used herein refers any substance having a measurable activity of therapeutic, cosmetic or nutritional nature, towards a human or animal to which the active ingredient is administered. The terms active ingredient and active agent are used interchangeably herein. One or more of numerous such active ingredients can be utilized in forming the products of this invention, including, for example, pharmaceuticals, dietary supplements, animal feeds, or biocidal agents. The processes according to the present invention are particularly useful for preparation of tablets comprising active drug substances or therapeutic agents, where such as formulations contain from 0.01% by weight up to about 99.9% by weight of the tablet, alternatively containing up to about 90%, about 80%, about 70%, about 60%, about 50%, about 40%, about 30%, about 20%, or about 10% by weight of the final dosage form.

Where the active ingredient is a pharmaceutical agent, representative general classifications of such agents include, for example, adrenergics; adrenocortical steroids; adrenocortical suppressants; aldosterone antagonists; amino acids; anabolics; analeptics; analgesics; anesthetics; anorectics; antiacne agents; antiadrenergics; antiallergics; antiamebics; antianemics; antianginals; antiarthritics; antiasthmatics; antiatherosclerotics; antibacterials; anticholinergics; anticoagulants; anticonvulsants; antidepressants; antidiabetics; antidiarrheals; antidiuretics; antiemetics; antiepileptics; antifibrinolytics; antifungals; antihemorrhagics; antihistamines; antihyperlipidemics; antihypertensives; antihypotensives; antiinfectives; antiinflammatories; antimicrobials; antimigraine; antimitotics; antimycotics, antinauseants, antineoplastics, antineutropenics, antiparasitics; antiproliferatives; antipsychotics; antirheumatics; antiseborrheics; antisecretories; antispasmodics; antithrombotics; antiulceratives; antivirals; appetite suppressants; blood glucose regulators; bone resorption inhibitors; bronchodilators; cardiovascular agents; cholinergics; depressants; diagnostic aids; diuretics; dopaminergic agents; estrogen receptor agonists; fibrinolytics; fluorescent agents; free oxygen radical scavengers; gastrointestinal motility effectors; glucocorticoids; hair growth stimulants; hemostatic agents; histamine H₂ receptor antagonists; hormones; hypocholesterolemics; hypoglycemics; hypolipidemics; hypotensives; imaging agents; immunizing agents; immunomodulators; immunoregulators; immunostimulants; immunosuppressants; keratolytics; LHRH agonists; mood regulators; mucolytics; mydriatics; nasal decongestants; neuromuscular blocking agents; neuroprotective agents; NMDA antagonists; non-hormopal sterol derivatives; plasminogen activators; platelet activating factor antagonists; platelet aggregation inhibitors; psychotropics; radioactive agents; scabicides; sclerosing agents; sedatives; sedative-hypnotics; selective adenosine Al antagonists; serotonin antagonists; serotonin inhibitors; serotonin receptor antagonists; steroids; thyroid hormones; thyroid inhibitors; thyromimetics; tranquilizers; amyotrophic lateral sclerosis agent; cerebral ischemia agent; Paget's disease agent; unstable angina agent; vasoconstrictor; vasodilator; wound healing agent; and antineoplastic agents. In a preferred embodiment of the invention, the composition contains Phenylephrine HCl.

Examples of analgesics include codeine, diamorphine, dihydromorphine, ergotamine, fentanyl and morphine; examples of antiallergics include cromoglycic acid and nedocromil; examples of antibiotics include cephalosporins, fusafungin, neomycin, penicillins, pentamidine, streptomycin, sulfonamides and tetracyclines; examples of anticholinergics include atropine, atropine methonitrate, ipratropium bromide, oxitropium bromide and trospium chloride; examples of antihistamines include H₁ or H₂ antagonists or other types of histamine release inhibitors, the H₁ antagonists can be sedating or non-sedating, such as diphenhydramine, chlorpheniramine, tripelennamine, promethazine, clemastine, doxylamine, astemizole, terfenadine, loratadine and desloratadine, among others, the H₂ antagonists include, but are not limited to, cimetadine, famotidine, nizatidine, and ranitidine; examples of histamine-release-inhibitors include, but are not limited to, cromolyn; examples of antiinflammatory substances include beclomethasone, budesonide, dexamethasone, flunisolide, fluticasone, tipredane and triamcinolone; examples of antitussives include narcotine and noscapine; examples of bronchodilators include bambuterol, bitolterol, carbuterol, clenbuterol, ephedrine, epinephrine formoterol, fenoterol, hexoprenaline, ibuterol, isoprenaline, isoproterenol, metaproterenol, orciprenaline, Phenylephrine HCl, pseudoephedrine, phenylpropanolamine, pirbuterol, procaterol, reproterol, rimiterol, salbutamol, salmeterol, sulfonterol, terbutalin and tolobuterol; examples of diuretics include amiloride and furosemide; examples of enzymes include amylase, lipase, protease and trypsin; examples of cardiovascular substances include diltiazem and nitroglycerine; examples of hormones include cortisone, hydrocortisone, prednisolone cyproterone acetate, norethisterone acetate, progesterone, 3-keto-desogestrel, norgestimate, laevonorgestrel, desogestrel, gestodene, estrogen, δ-4-androstenedione, testosterone, dihydrotestosterone, or androstanolone, examples of proteins and peptides include cyclosporins, cetrorelix, glucagon and insulin.

Example classes of antineoplastic agents include, but are not limited to, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Particularly useful members of those classes include, for example, doxorubicin, caminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin or podophyllotoxin derivatives such as colchicines, etoposide, etoposide phosphate or teniposide, vinblastine, vincristine, leurosidine, vindesine, leurosine, and the like. Other useful antineoplastic agents include estramustine, cisplatin, carboplatin, cyclophosphamide, bleomycin, tamoxifen, ifosamide, melphalan, hexamethyl melamine, thiotepa, cytarabine, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

Suitable anti-inflammatory and/or antipyretic agents useful for the present compositions may be: a non-steroidal anti-inflammatory (NSAIDs), aminoarylcarboxylic acid derivatives such as enfenamic acid, etofenamate, flufenamic acid, isonixin, meclofenamic acid, mefanamic acid, niflumic acid, talniflumate, terofenamate and tolfenamic acid; arylacetic acid derivatives such as acemetacin, alclofenac, amfenac, bufexamac, cinmetacin, clopirac, diclofenac sodium, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiaramide, tolmetin and zomepirac; arylbutyric acid derivatives such as bumadizon, butibufen, fenbufen and xenbucin; arylcarboxylic acids such as clidanac, ketorolac and tinoridine; arylpropionic acid derivatives such as alminoprofen, benoxaprofen, bucloxic acid; carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen and tiaprofenic acid; pyrazoles such as difenamizole and epirizole; pyrazolones such as apazone, benzpiperylon, feprazone, mofebutazone, morazone, oxyphenbutazone, phenybutazone, pipebuzone, propyphenazone, ramifenazone, suxibuzone and thiazolinobutazone; salicylic acid derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate and sulfasalazine; thiazinecarboxamides such as droxicam, isoxicam, piroxicam and tenoxicam; others such as ÿ-acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole and tenidap; and pharmaceutically acceptable salts thereof; and other analgesics, such as acetaminophen.

In preferred embodiments the compositions of the invention may contain a decongestant. In other preferred embodiments, the compositions of the invention may contain an antihistamine, in particular loratadine or desloratadine.

Nutritionally active agents can include typical dietary supplements such as vitamins, minerals, proteins, and the like. Examples of nutritional actives include, but are not limited to, Vitamin A, Vitamin B-1, Vitamin B-2, Vitamin B-3, Vitamin B-6, Vitamin B-12 Vitamin C, Vitamin D3, Vitamin E, Vitamin K, biotin folic acid, pantothenic acid, boron, calcium, choline, chromium, copper, iodine, magnesium, manganese, molybdenum, potassium, selenium, zinc, cranberry powder, black cohosh root extract, chastetree berry extract, green tea leaf extract, lycopene, milk thistle extract, panax ginseng root extract, saw palmetto berry extract, turmeric extract, malic acid, methylsulfonylmethane, trimethylglycine, alpha-lipoic acid, calcium-d-glucarate, N-acetyl cysteine, and inulin.

As used herein, the term cosmetically active ingredients refer more particularly to compounds or compositions that when ingested have an effect on the outward appearance of the skin or hair of a subject. Such ingredients may include active ingredients listed above as pharmaceutically active ingredients or nutritionally active ingredients. Additional ingredients that may also be considered cosmetically active ingredients may include wetting agents; depigmenting agents such as hydroquinone, azelaic acid, caffeic acid or kojic acid; emollients; moisturizing agents such as glycerol, PEG 400, thiamorpholinone and its derivatives or alternatively urea; antiseborrhoeic or antiacne agents such as S-carbo-xymethylcysteine, S-benzylcysteamine, salts or derivatives thereof, benzoyl peroxide; agents promoting hair regrowth, such as Minoxidil (2,4-diamino-6-piperidinopyrimidine 3-oxide) and derivatives thereof, Diazoxide (7-chloro-3-methyl-1,2,4-benzothiadiazime-1,1-dioxide) and Phenyloin (5,4-diphenyl-2,4-imidazolidinedione).

In other embodiments, the compositions of the invention may comprise additional pharmaceutically, cosmetically or nutritionally active ingredients that may or may not contain primary or secondary amines in combination with the active ingredients having a primary or secondary amine moiety. These additional active ingredients may fall under the same general classifications and specific examples noted above.

The active ingredients mentioned by way of example can be employed as free bases or acids or as pharmaceutically acceptable salts. Counterions which can be employed are, for example, physiologically tolerable alkaline earth metals or alkali metals or amines, as well as, for example, acetate, benzenesulfonate, benzoate, hydrogen carbonate, hydrogen tartrate, bromide, chloride, iodide, carbonate, citrate, fumarate, malate, maleate, gluconate, lactate, pamoate and sulfate. Esters can also be employed, for example including but not limited to acetate, acetonide, propionate, dipropionate, valerate.

Compositions of the invention containing the active coating composition defined herein can be formulated as any form convenient for administration to subject in need thereof. Typical forms include tablets, caplets, pill, hard or soft capsule, lozenge, powder, granule, a suspension, a liquid. Preferably, the composition is in a form suitable for oral dosage that are discrete dose units each containing a predetermined amount of the pharmaceutically active agent, such as tablets or capsules.

Further, the compositions of the invention can comprise other excipients used in formulation of pharmaceutical compositions provided that they do not add appreciable amounts of cellulosic materials to the active coating dispersion's composition of the invention. As used herein, the term excipient refers to any substance that is not a therapeutic agent but is added to a composition to improve handling or storage properties or to aid in manufacture of a dosage forms of the pharmaceutical composition. Examples include but are not limited to substances commonly referred to as diluents, disintegrants, binding agents, adhesives, wetting agents, lubricants, glidants, crystallization inhibitors, viscosity modifying agents, surface modifying agents, emulsifiers, film forming agents, plasticizers, pH modifying agents, taste or odor modifying or masking agents, flavors, sweeteners, dyes, and fragrances.

If desired, a finish coating can also be applied to the composition coated with the active ingredient. In certain embodiments one or more colorants can be formulated into this finish coat as desired. Colorant and other additives or processing aids could also be formulated into the active ingredient coat if desired.

### Exemplary Coating Composition Formulations and Methods

### Phenylephrine HCl in PVA-Based Coating Composition.

In one embodiment of this invention an aqueous dispersion can be formed containing Phenylephrine HCl and an PVA-based coating composition such as Opadry® II 85F18422 supplied by Colorcon®. In the practice of this aspect of the invention, PE is not limited to any particle size and may be present in an amount between about 0.1% to about 99.9% w/w of total solids added to the dispersion, and optimally is formulated so the solids level for Phenylephrine HCl is 7.5 mg/tablet (16-18% w/w of total solids added into the dispersion). The Opadry® II 85F18422 system may be present in an amount between about 0.1% to about 99.9% w/w of total solids added to the dispersion, and optimally is formulated at about 36 mg/tablet (82-84% w/w of the final solids in the solution). This Opadry® II level could be higher, for example exceeding 50 mg/tablet, if desired. The PVA-based coating composition optimally contains additional processing aids such as PEG 3350, talc and titanium dioxide, but could also contain additional film forming agents, colorants, opacifiers, plasticizers, surfactants, anti-foam agents and detackifiers. Optimally, solids content for the dispersion would range from 5-25% (w/w), but could range from 0.1-50% w/w and mixed with purified water optimally or any appropriate solvent.

Methods of formulating the compositions are known to those of ordinary skill in the art. Order of addition for this formulation does not matter as the drug and polymer are both highly water soluble. Moderate agitation is preferably maintained to avoid excess foaming. Moderate mixing is preferably performed for approximately 45 minutes to 1 hour after final addition of Opadry® II blend. Reducing mixing speed prior to spraying to de-gas the system is also recommended.

The aqueous dispersion can then be sprayed onto a substrate such as a tablet optimally where spraying is initiated when an exhaust temperature of between 45° C. to 55° C., preferably at a temperature range of between about 48° C. to 50° C. Slower spray rates would optimally provide the best content uniformity as it increases the instances a certain tablet is exposed to the coating spray stream in an overall coating run. In the practice of this invention where the formulation is intended to be coated on a tablet surface, no sub-coat is needed in this system to act as a chemical or physical barrier to maintain optimal PE chemical stability. However, a sub-coat consisting of any material could be applied if it was determined to be of aesthetic or chemical importance. Further if desired a finish coat consisting of any material can be applied but will optimally contain a similar PVA-based system such as Opadry® II white 85F18422. The finish coat can be applied at a level of 10-15 mg/tablet and formulated with water or other appropriate solvents at a solids content of 10-25% w/w, more specifically with a solids content of 15-20% w/w as recommended by the manufacturer, or preferably with a solids content of 15.25% w/w. However, the solids content could vary based on processing conditions.

### Phenylephrine HCl and Loratadine in PVA-Based Coating Compositions.

In another embodiment of the invention, an antihistamine such as loratadine can be added to the dispersion and coated to a substrate. The level of loratadine can be between 0.1 and 99% w/w of total solids added into the dispersion and is optimally present at a level of 5 mg/tablet. In the practice of this aspect of the invention, the solids level for Phenylephrine HCl is 7.5 mg/tablet (14-16% w/w of total solids added into dispersion), Loratadine is 5 mg/tablet (9-11% w/w of total solids added into dispersion) and the Opadry® II 85F18422 is 36 mg/tablet (73-75% w/w of total solids added into dispersion). This Opadry® II level could be higher (≧54 mg/tablet) if desired. Opadry® II 85F18422 would optimally contain a polyalkylene glycol such as PEG 3350, talc and titanium dioxide, and may also contain additional colorants, opacifiers, plasticizers, surfactants, film forming agents, anti-foam agents and detackifiers at levels determined by those of ordinary skill in the art. Levels of drug to polymer and or polymer composition or polymer system for this aspect of the invention may range from 0.1-99.9% (w/w of total solids added into dispersion) of Phenylephrine HCl, 0.1%-99.9% (w/w of total solids added into dispersion) of loratadine and 0.1-99.9% (w/w of total solids added into dispersions) of Opadry® II 85F series, Opadry® II 85G series or Opadry® AMB® (PVA based coating system). Optimally, solids content for the dispersion would range from 5-25% (w/w), but could range from 0.1-50% w/w and mixed with purified water optimally or other appropriate solvents.

Methods of formulating the compositions are known to those of ordinary skill in the art. Order of addition of components for this formulation is important if an active agent having limited solubility such as loratadine is incorporated into the formulation. As noted above, Phenylephrine HCl and the PVA-based polymer blend are both highly water soluble, but it is preferred that Phenylephrine HCl is added first followed by the PVA-based polymer blend as Phenylephrine HCl is more soluble. Moderate agitation preferably should be maintained to avoid excess foaming. Moderate mixing preferably should be performed for approximately 45 minutes to 1 hour after final addition of the PVA-based polymer blend.

It is recommended that loratadine or similar low solubility active agent then be added to the mixing vessel and stirred at moderate agitation until all of the active agent appeared wetted and incorporated into the suspension. In the formulation of examples according to the invention it was noted that the PVA based Opadry® II polymer system has some surface activity when dispersed and enhances the wettability of the more water insoluble ingredients. Further, in the formulation of examples according to the invention it was noted that the loratadine does disperse to some degree, but agglomerates in the millimeter size range are observed in the milky white suspension. In a preferred embodiment, the vessel containing the formulation is preferably then transferred to a high shear mixer for further particle size reduction of the water insoluble components such as loratadine. This could also be accomplished through an inline high shear apparatus for a short period of time, approximately 3 to 5 minutes. In the formulation of examples according to the invention, after high shear mixing all individual drug and excipient particles appeared discrete and dispersed when viewed under a microscope.

Coating methods discussed above can also be applied to the formulation containing the additional active agent such as loratadine. Slower spray rates would optimally provide the best content uniformity as it increases the instances a certain tablet is exposed to the coating spray stream in an overall coating run. If this formulation is intended to be coated on a tablet surface, no sub-coat is needed in this system to act as a chemical or physical barrier to maintain optimal PE chemical stability. As noted above, a sub-coat consisting of any material could be applied if it was determined to be of aesthetic or chemical importance.

### Phenylephrine HCl in PVA Derived Copolymer Based Formulation.

In the practice of this aspect of the invention, a dispersion can be formed comprising Phenylephrine HCl and a PVA-copolymer composition. A preferred PVA-copolymer composition are those such as PVA-polyalkylene glycol copolymers such as Kollicoat® IR which is a PVA-PEG 3350 (75/25% w/w) graft co-polymer produced by BASF. In the practice of this aspect of the invention, levels of drug to polymer for this system could range from 0.1-99.9% of Phenylephrine HCl (w/w) or 0-60 mg/tablet and 0.1-99.9% (w/w) Kollicoat® IR. Optimally, final solids content of the dispersion would be 10-25% (w/w), but could range from 0.1-50% (w/w). The dispersions are preferably formed with purified water or other appropriate solvents. According to this aspect of the invention; the dispersion is optimally formulated so the solids level for Phenylephrine HCl is 7.5 mg/tablet (16-18% w/w of total solids added into dispersion) and the Kollicoat® IR is 36 mg/tablet (82-84% w/w of total solids added into dispersion) of the final solids in the solution. In the practice of this aspect of the invention the dispersions do not contain any additional excipients. However additional colorants, opacifiers, plasticizers, surfactants, viscosity modifying agents, pH modifying agents, anti-foam agents and detackifiers could be added as desired by one of ordinary skill in the art. Antifoam agents at levels of 0.01-5.0% may be needed to reduce foaming when mixing.

Similar to the example noted above for Phenylephrine HCl PVA-based coating compositions, the order of addition of components for this formulation does not matter as Phenylephrine HCl and the PVA-copolymer blend are both highly water soluble. Temperature for coating should be maintained at 46-49° C. for materials produced according to this aspect of the invention. Optimally, solution would be sprayed onto tablet optimally where spraying is initiated when an exhaust temperature of 50° C. is achieved. In the preparation of examples according to this aspect of the invention, exhaust temperatures as low as 42-44° C. were used successfully under very slow spray rates. Slower spray rates may optimally provide the best content uniformity as it increases the instances a certain substrate such as a tablet is exposed to the coating spray stream in an overall coating run. In the practice of this aspect of the invention where the dispersion is intended to be coated on a tablet surface, no sub-coat is needed to act as a chemical or physical barrier to maintain optimal Phenylephrine HCl chemical stability. However a sub-coat consisting of any material could be applied if it was determined to be of aesthetic or chemical importance. Moreover a finish coat can also be applied if desired. The finish coat can be applied at a level of 10-15 mg/tablet and formulated with water or other appropriate solvents at a solids content of 10-25% w/w, more specifically with a solids content of 15-20% w/w as recommended by the manufacturer, or preferably with a solids content of 15.25% w/w. However, the solids content could vary based on processing conditions.

### Phenylephrine HCl and Loratadine in PVA-Copolymer Based Coating Compositions.

In another embodiment of the invention, an antihistamine such as loratadine can be added to the dispersion and coated to a substrate. The level of loratadine can be between 0.1 and 20% (w/w of total solids in the final dosage form) and is optimally present at a level of 5 mg/tablet. In the practice of this aspect of the invention, the solids level for Phenylephrine HCl is 7.5 mg/tablet (14-16% w/w of total solids added into dispersion), Loratadine is 5 mg/tablet (9-11% w/w of total solids added into dispersion) and the Kollicoat® IR is 36 mg/tablet (73-75% w/w of total solids added into dispersion). This dispersion may also contain additional excipients including; colorants, opacifiers, plasticizers, viscosity modifying agents, film forming agents, pH modifying agents, surfactants, emulsifiers, anti-foam agents, colorants, taste modifying or masking agents and detackifiers at levels determined by those of ordinary skill in the art. Levels of active agent to PVA copolymer for this aspect of the invention may range from 0.1-99.9% (w/w) of Phenylephrine HCl, 0.1%-99.9% (w/w) of loratadine and 0.1-99.9% (w/w) of Kollicoat® IR in the dispersion. Optimally solids content for the dispersion would range from 5-25% (w/w), but could range from 0.1-50% w/w and mixed with purified water optimally or other appropriate solvents.

Methods of formulating the compositions are known to those of ordinary skill in the art. Order of addition of components for this formulation is important if an active agent having limited solubility such as loratadine is incorporated into the formulation. As noted above, Phenylephrine HCl and the PVA-copolymer based polymer blend are both highly water soluble and should be added first to the dispersion. Moderate agitation preferably should be maintained to avoid excess foaming. Moderate mixing should be performed for at least 1 hour after the final addition of the Kollicoat® IR or until a translucent solution is formed.

It is recommended that loratadine or similar low solubility active agent then be added to the mixing vessel and stirred at moderate agitation until all of the active agent appeared wetted and incorporated into the suspension. In the formulation of examples according to the invention it was noted that the PVA maintains some surface activity and enhances the wettability of the more water insoluble ingredients. Further, in the formulation of examples according to the invention it was noted that the loratadine does disperse to some degree, but agglomerates in the millimeter size range are observed in the milky white suspension. In a preferred embodiment the vessel containing the formulation is then transferred to a high shear mixer for further particle size reduction of the water insoluble components such as loratadine. This could also be accomplished through an inline high shear apparatus for a short period of time, approximately 3 to 5 minutes. In the formulation of examples according to the invention, after high shear mixing all individual drug and excipient particles appeared discrete and dispersed when viewed under a microscope.

Coating methods discussed above for Phenylephrine HCl in PVA-copolymer based coating compositions can also be applied to the formulation containing the additional active agent such as loratadine. Slower spray rates would optimally provide the best content uniformity as it increases the instances a certain tablet is exposed to the coating spray stream in an overall coating run. If this formulation is intended to be coated on a tablet surface, no sub-coat is needed in this system to act as a chemical or physical barrier to maintain optimal Phenylephrine HCl chemical stability. As noted above, a sub-coat consisting of any material could be applied if it was determined to be of aesthetic or chemical importance.

In the preparation of examples according to this aspect of the invention containing loratadine, the loratadine was seen to be dispersed into primary particles which could be maintained as long as moderate shear is maintained in the aqueous system during the entire coating cycle.

The following examples are intended to be illustrative of the disclosed invention. The examples are non-limiting, and the skilled artisan will recognize that other embodiments are within the scope of the disclosed invention.

### EXAMPLES

Examples 1-3 show exemplary formulations for tablet coatings providing for immediate release of phenylephrine HCl, loratadine (containing secondary amine moieties), and epinastine (containing primary amine moiety). The formulations use PVA based systems such as the Kollicoat® IR or Opadry® II 85F 18422 (white) in the active coat as well as for the Opadry® white 85F 18422 used in the finish coating dispersion. The formulations are prepared according to similar methods as described above.

### Example 1

### Active Ingredient Dispersion Using PVA Based Polymer System (Opadry® II 85F18422) and Free of Cellulosic Material and Comprising Phenylephrine and Loratadine

| Drug/Coating layer | Component | weight (mg/tablet) |
|---|---|---|
| Active Dispersion | Water | Adjusted to achieve |
| (≦23% solids w/w) | | desired solids |
| | | concentration |
| | Opadry ® II 85F18422 | 36.0 |
| | Phenylephrine HCl | 7.5 |
| | Loratadine | 5.0 |
| Finish Coating | Water | Adjusted to achieve |
| (≦20% solids w/w) | | desired solids |
| | | concentration |
| | Opadry ® II 85F18422 | 36.0 |

### Example 2

### Active Ingredient Dispersion Using PVA Based Polymer System (Opadry® II 85F18422) and Free of Cellulosic Material and Comprising Epinastine

| Drug/Coating layer | Component | weight (mg/tablet) |
|---|---|---|
| Active Dispersion | Water | Adjusted to achieve |
| (≦23% solids w/w) | | desired solids |
| | | concentration |
| | Opadry ® II 85F18422 | 36.0 |
| | Epinastine | 20.0 |
| Finish Coating | Water | Adjusted to achieve |
| (≦20% solids w/w) | | desired solids |
| | | concentration |
| | Opadry II 85F18422 | 36.0 |

### Example 3

### Active Ingredient Dispersion Using PVA/PEG 3350 Graft Co-Polymer System (Kollicoat® IR) and Free of Cellulosic Material and Comprising Phenylephrine and Loratadine

| Drug/Coating layer | Component | weight (mg/tablet) |
|---|---|---|
| Active Dispersion | Water | Adjusted to achieve |
| (≦23% solids w/w) | | desired solids |
| | | concentration |
| | Kollicoat ® IR | 36.0 |
| | Phenylephrine HCl | 7.5 |
| | HCl | |
| | Loratadine | 5.0 |
| Finish Coating | Water | Adjusted to achieve |
| (≦20% solids w/w) | | desired solids |
| | | concentration |
| | Opadry ® II 85F18422 | 36.0 |

The dispersions of Examples 1-3 can be applied to a tablet substrate (e.g., 500 mg/tablet) using methods as described above. Table 1 provides the preferred coating process parameters. Values in parenthesis are the operational ranges successfully used to achieve acceptable coating utilizing an O'Hara Labcoat® MX 12″ coating pan. Similar results were also produced in Vector LDCS® 12″, Accela® 24″, Accela® 48″ and Accela® 60″ coating pans.

TABLE 1
Tablet coating parameters

| | | Active coat | Finish coat |
|---|---|---|---|
| | | using | using |
| | Active coat | Opadry ® II | Opadry ® II |
| | using | 85F18422 | 85F18422 |
| Coating Parameter | Kollicoat ® IR | (white) | (white) |
| *Solids content in solution | 18.7% | 18.7% | 15.25% |
| (w/w) | | | |
| Target weight gain | 48.5 | 48.5 | 10-15 |
| (mg/tablet) | | | |
| Exhaust Temp (approx.) | 46-49 | 48-50 | 48-50 |
| (° C.) | (40-55) | (45-55) | (45-55) |
| Inlet Temp (° C.) | 68 (60-75) | 65 (60-70) | 65 (60-70) |
| *Spray rate (approx.) | 3.5-6.5 | 3.5-5.5 | 3.5-5.5 |
| g/min*kg | | | |
| Air volume (cfm) | 100 (80-120) | 100 (80-120) | 100 (80-120) |
| Atomization Air Pressure | 20 (15-25) | 20 (15-25) | 20 (15-25) |
| (psi) | | | |
| Pattern Air Pressure | 18 (15-25) | 18 (15-25) | 18 (15-25) |
| pan speed (rpm) | 20 (16-20) | 20 (16-20) | 20 (16-20) |
| Desired final batch weight | 0.8-0.9 | 0.8-0.9 | 0.8-0.9 |
| (kg) | | | |

*Can be reduced to if necessary to increase spray time and minimize variability of the drug substance content uniformity in the tablet film.

### Example 4

### Phenylephrine HCl Stability Study

Methods: Chemical compatibility of Phenylephrine HCl with various film forming polymers or polymer blends and plasticizers (Table 2) was conducted. Five test formulations (A, B, D, E, G) containing approximately 200-350 grams of each aqueous dispersion were prepared. Two additional formulations (C and F) were used as controls. Sample C contained Phenylephrine HCl dissolved in water without any additional cellulosic or non-cellulosic polymers. Sample F comprised phenylephrine HCl in the absence of water. Both controls were subjected to the same drying conditions as the other example films prior to analysis.

TABLE 2

| Formulation | | | | | | |
|---|---|---|---|---|---|---|
| A | B | ^{(a)}C | D | E | ^{(b)}F | G |

(mg per film equivalent to 10 unit doses of the active)

| | | | | | | | |
|---|---|---|---|---|---|---|---|
| HPMC | | | | 146 | | | |
| (Hydroxypropyl | | | | | | | |
| methylcellulose - | | | | | | | |
| grade E5) | | | | | | | |
| PVA based - | 183 | 183 | | | | | |
| Opadry ® II | | | | | | | |
| 85G90706 (with Blue | | | | | | | |
| #1 color) | | | | | | | |
| HPC (Hydroxypropyl | | | | | 146 | | |
| Cellulose - grade EF) | | | | | | | |
| BHA (Butylated | | 2 | | | | | |
| Hydoxyanisole) | | | | | | | |
| Polyethylene Glycol | | | | | | | 40 |
| (PEG) 3350 | | | | | | | |
| Phenylephrine HCl | 75 | 75 | 75 | 75 | 75 | 75 | 75 |
| Water (evaporated | 2146 | 2161 | 3247 | 3245 | 3245 | 0 | 3238 |
| during drying) | | | | | | | |
| Total solids per film | 258 | 260 | 75 | 221 | 221 | 75 | 115 |
| Total film weight | 2404 | 2421 | 3322 | 3466 | 3466 | 75 | 3353 |
| Percentage of total | 0.18 | 0.20 | 0.38 | 2.12 | 1.44 | 0.02 | 0.35 |
| PE degradation | | | | | | | |
| products per film | | | | | | | |

^{(a)}PE and water only dispersion. PE dried like other films. Tacky, non-crystalline material resulted after drying.
^{(b)}PE not dispersed in water. Crystalline PE dried and stored in same conditions as films.

Aliquots from the bulk coating dispersion equivalent to approximately 10 unit doses of the active were poured into flat, 4-6″ plastic weight boats and allowed to dry overnight for approximately 15-16 hours at 40C under vacuum. The amount per film equivalent to 10 unit doses was chosen for achieving reproducible and reliable analytical results for the Phenylephrine HCl degradants. These dried film and active drug samples were then removed from drying when no significant change in mass had been observed. These dried film samples were then stored at 50C in closed amber jars for 14 days. Each film was then analyzed by HPLC for the presence of degradation products. Separation of Phenylephrine HCl degradation products was accomplished on an HPLC using a Prontosil EPS C18, 100×4.6 mm (3 μparticle size) column manufactured by Bischoff Chromatography and using a gradient test method. Detection was by UV at 215 nm. The column was thermostated at 25° C. and an injection volume of 10 μL of sample solution was used. Known and unknown impurities were reported as peak area percent using data acquisition software.

Results: Samples C and F are controls. Degradation for Samples C and F are compared to samples A, B, D, E and G to determine the effects of the coating substances on the degradation of Phenylephrine hydrochloride (PE). For Sample F (PE only), no appreciable degradation occurred (0.02% total PE degradants). For sample C (PE and water), the total PE degradant amount found was 0.38%. Additionally, Sample C (PE and water) retained 10-15% residual water after drying. Residual water after drying of the film samples may vary because water retention is a material property and varies among formulations. Samples A and B containing PVA and Phenylephrine HCl showed total phenylephrine HCl degradation levels less than the control sample C, which may demonstrate a stabilizing affect of PVA polymers on PE. In comparison, samples D and E containing cellulosic polymers (HPMC and HPC) with PE exhibited significant total PE degradation formation. These degradation levels are approximately 3.5 to 5 times greater than Sample C control, demonstrating incompatibility of cellulosic polymers with PE. Sample G, containing phenylephrine HCl and PEG 3350 (plasticizer) in an aqueous solution produced a degradation level nearly identical to the Sample C control, demonstrating compatibility with phenylephrine HCl.

### Example 5

### Desloratadine Stability Study

Methods: Chemical compatibility of desloratadine with various film forming polymers or polymer blends and plasticizers (Table 3) was conducted. Three test formulations (A, B, C) were prepared similar as described above in Example 4 but containing desloratadine in place of phenylephrine HCl. Two additional formulations were prepared as controls, desloratadine and water only dispersion (D) and crystalline desloratadine not dispersed in water (E). All samples and controls were dried and stored in same conditions prior to analysis.

TABLE 3

| | Formulation | | | | |
|---|---|---|---|---|---|
| | A | B | C | ^{(a)}D | ^{(b)}E |
| (mg per film equivalent to 10 unit doses of the active) | | | | | |
| HPMC (Hydroxypropyl | 146 | | | | |
| memylcellulose - | | | | | |
| grade E5) | | | | | |
| PVA based - Opadry ® | | | 183 | | |
| II 85G90706 (with Blue | | | | | |
| #1 color) | | | | | |
| HPC (Hydroxypropyl | | 146 | | | |
| Cellulose - grade EF) | | | | | |
| Desloratadine | 50 | 50 | 50 | 50 | 50 |
| Water (evaporated in | 3060 | 3060 | 1938 | 2874 | 0 |
| the drying process) | | | | | |
| Total solids per film | 196 | 196 | 233 | 50 | 50 |
| Total film weight | 3206 | 3206 | 2120 | 2924 | 50 |
| Percentage of total | 0.00 | 0.33 | 0.06 | 0.00 | 0.00 |
| Desloratadine | | | | | |
| degradation | | | | | |
| products per film | | | | | |

^{(a)}Desloratadine and water only dispersion. Desloratadine dried like other films. Dry white film formed.
^{(b)}Desloratadine not dispersed in water. Crystalline desloratadine dried and stored in same conditions as films

Aliquots from the bulk coating dispersion equivalent to approximately 10 unit doses of the active were poured into flat, 4-6″ plastic weight boats and allowed to dry overnight for approximately 15-16 hours at 40C under vacuum. The amount per film equivalent to 10 unit doses was chosen for achieving reproducible and reliable analytical results for the desloratadine degradants. These dried film and active drug samples were then removed from drying when no significant change in mass had been observed. These dried film samples were then stored at 50C in closed amber jars for 14 days. Each film was then analyzed by HPLC for the presence of degradation products. Separation of desloratadine degradation products was accomplished on an HPLC using a TMC-Pack Pro C18 column (3μ particle size) column manufactured by YMC Co. Ltd. and using a gradient test method. Detection was by UV at 267 nm. The column was thermostated at 25° C. and an injection volume of 20 μL of sample solution was used. Known and unknown impurities were reported as peak area percent using data acquisition software.

Results: Desloratadine dispersed and applied in a PVA containing film (Sample C) produced significantly less chemical degradation than desloratadine dispersed and applied in a hydroxypropyl cellulose containing film (Sample B). Although not wishing to be bound by theory, it is believed that HPMC in Sample A reacts differently with desloratadine than HPC (and it, is believed, other cellulosic polymers) resulting in no detectable desloratadine degradation products.

Several embodiments of the present invention are specifically described herein. However, it will be appreciated that modifications and variations of the present invention are covered by the above teachings and within the purview of the appended claims without departing from the spirit and intended scope of the invention.

What is claimed is:

1. A method of manufacturing a composition of matter for consumption by an animal, wherein the composition of matter comprises a compound containing a primary or secondary amine moiety, the method comprising the step of coating a consumable substrate with a coating composition comprising said compound, wherein said coating composition does not contain an appreciable amount of cellulosic materials.

2. The method of claim 1, wherein the compound containing a primary or secondary amine is a pharmaceutically active ingredient.

3. The method of claim 2, wherein the pharmaceutically active ingredient is chosen from the group consisting of adrenergics; adrenocortical steroids; adrenocortical suppressants; aldosterone antagonists; amino acids; anabolics; analeptics; analgesics; anesthetics; anorectics; antiacne agents; antiadrenergics; antiallergics; antiamebics; antianemics; antianginals; antiarthritics; antiasthmatics; antiatherosclerotics; antibacterials; anticholinergics; anticoagulants; anticonvulsants; antidepressants; antidiabetics; antidiarrheals; antidiuretics; antiemetics; antiepileptics; antifibrinolytics; antifungals; antihemorrhagics; antihistamines; antihyperlipidemics; antihypertensives; antihypotensives; antiinfectives; antiinflammatories; antimicrobials; antimigraine; antimitotics; antimycotics, antinauseants, antineoplastics, antineutropenics, antiparasitics; antiproliferatives; antipsychotics; antirheumatics; antiseborrheics; antisecretories; antispasmodics; antithrombotics; antiulceratives; antivirals; appetite suppressants; blood glucose regulators; bone resorption inhibitors; bronchodilators; cardiovascular agents; cholinergics; depressants; diagnostic aids; diuretics; dopaminergic agents; estrogen receptor agonists; fibrinolytics; fluorescent agents; free oxygen radical scavengers; gastrointestinal motility effectors; glucocorticoids; hair growth stimulants; hemostatic agents; histamine H₂ receptor antagonists; hormones; hypocholesterolemics; hypoglycemics; hypolipidemics; hypotensives; imaging agents; immunizing agents; immunomodulators; immunoregulators; immunostimulants; immunosuppressants; keratolytics; LHRH agonists; mood regulators; mucolytics; mydriatics; nasal decongestants; neuromuscular blocking agents; neuroprotective agents; NMDA antagonists; non-hormonal sterol derivatives; plasminogen activators; platelet activating factor antagonists; platelet aggregation inhibitors; psychotropics; radioactive agents; scabicides; sclerosing agents; sedatives; sedative-hypnotics; selective adenosine Al antagonists; serotonin antagonists; serotonin inhibitors; serotonin receptor antagonists; steroids; thyroid hormones; thyroid inhibitors; thyromimetics; tranquilizers; amyotrophic lateral sclerosis agent; cerebral ischemia agent; Paget's disease agent; unstable angina agent; vasoconstrictor; vasodilator; wound healing agent; xanthine oxidase inhibitor; and antineoplastic.

4. The method of claim 2, wherein the pharmaceutically active ingredient is Phenylephrine HCl.

5. The method of claim 1, wherein the compound containing a primary or secondary amine is a cosmetically active ingredient.

6. The method of claim 1, wherein the compound containing a primary or secondary amine is a nutritionally active ingredient.

7. The method of claim 1 wherein the coating composition comprises said compound containing a primary or secondary amine moiety and polyvinyl alcohol.

8. The method of claim 1, wherein the coating composition consists essentially of said compound containing a primary or secondary amine moiety and polyvinyl alcohol.

9. The method of claim 1, wherein the coating composition consists essentially of said compound containing a primary or secondary amine moiety and a polyvinyl alcohol derived copolymer.

10. The method of claim 1, wherein the compositions further comprise excipients that do not add appreciable amounts of cellulosic materials to the coating composition.

11. The method of claim 10, wherein the excipient is chosen from the group consisting of diluents, disintegrants, binding agents, adhesives, wetting agents, lubricants, glidants, crystallization inhibitors, surface modifying agents, taste or odor modifying or masking agents, flavors, dyes, and fragrances.

12. The method of claim 1, wherein a finish coating is applied to the composition of matter.

13. The method of claim 1, wherein the composition of matter is in the form chosen from the group consisting of tablet, caplet, pill, hard capsule, soft capsule, lozenge, powder, granule, suspension, and liquid.

14. The method of claim 2, wherein the composition of matter is in discrete dose units containing a predetermined amount of the pharmaceutically active ingredient.

15. A pharmaceutical composition comprising a pharmaceutically active agent seated on a substrate, wherein the pharmaceutically active agent comprises a compound containing a primary or secondary amine moiety and the pharmaceutical composition does not contain an appreciable amount of cellulosic material.

16. A pharmaceutical composition comprising a pharmaceutically active agent seated on a substrate, wherein the pharmaceutically active agent comprises a compound containing a primary or secondary amine moiety and wherein the pharmaceutically active agent is applied to the substrate as part of a composition that does not contain an appreciable amount of cellulosic materials.

17. The composition of claim 16, wherein the immediate release coating is applied to the substrate as an aqueous solution or dispersion of the therapeutically active agent and the polyvinyl alcohol or polyvinyl alcohol derived copolymer.

18. A pharmaceutical composition comprising an immediate release coating composition on a substrate, the coating comprising a therapeutically active agent containing at least one primary or secondary amine moiety, the immediate release coating consisting essentially of the therapeutically active agent and polyvinyl alcohol or polyvinyl alcohol derived copolymer.

19. A pharmaceutical composition comprising an immediate release coating composition on a substrate, the coating comprising a therapeutically active agent containing at least one primary or secondary amine moiety, the immediate release coating consisting of the therapeutically active agent and polyvinyl alcohol or polyvinyl alcohol derived copolymer.

20. A method of manufacturing a composition of matter comprising applying to a substrate a coating composition comprising an active ingredient and a film forming agent, wherein the film forming agent comprises a polyvinyl alcohol or a polyvinyl alcohol derived copolymer, wherein the coating composition provides for immediate release of the pharmacologically or therapeutically active agent.

21. The method of claim 20, wherein the coating composition consists essentially of the active ingredient and polyvinyl alcohol or a polyvinyl alcohol derived copolymer.

22. The method of claim 20, wherein the active ingredient is a pharmaceutically active ingredient.

23. The method of claim 22, wherein the pharmaceutically active ingredient is chosen from the group consisting of adrenergics; adrenocortical steroids; adrenocortical suppressants; aldosterone antagonists; amino acids; anabolics; analeptics; analgesics; anesthetics; anorectics; antiacne agents; antiadrenergics; antiallergics; antiamebics; antianemics; antianginals; antiarthritics; antiasthmatics; antiatherosclerotics; antibacterials; anticholinergics; anticoagulants; anticonvulsants; antidepressants; antidiabetics; antidiarrheals; antidiuretics; antiemetics; antiepileptics; antifibrinolytics; antifungals; antihemorrhagics; antihistamines; antihyperlipidemics; antihypertensives; antihypotensives; antiinfectives; antiinflammatories; antimicrobials; antimigraine; antimitotics; antimycotics, antinauseants, antineoplastics, antineutropenics, antiparasitics; antiproliferatives; antipsychotics; antirheumatics; antiseborrheics; antisecretories; antispasmodics; antithrombotics; antiulceratives; antivirals; appetite suppressants; blood glucose regulators; bone resorption inhibitors; bronchodilators; cardiovascular agents; cholinergics; depressants; diagnostic aids; diuretics; dopaminergic agents; estrogen receptor agonists; fibrinolytics; fluorescent agents; free oxygen radical scavengers; gastrointestinal motility effectors; glucocorticoids; hair growth stimulants; hemostatic agents; histamine H₂ receptor antagonists; hormones; hypocholesterolemics; hypoglycemics; hypolipidemics; hypotensives; imaging agents; immunizing agents; immunomodulators; immunoregulators; immunostimulants; immunosuppressants; keratolytics; LHRH agonists; mood regulators; mucolytics; mydriatics; nasal decongestants; neuromuscular blocking agents; neuroprotective agents; NMDA antagonists; non-hormonal sterol derivatives; plasminogen activators; platelet activating factor antagonists; platelet aggregation inhibitors; psychotropics; radioactive agents; scabicides; sclerosing agents; sedatives; sedative-hypnotics; selective adenosine Al antagonists; serotonin antagonists; serotonin inhibitors; serotonin receptor antagonists; steroids; thyroid hormones; thyroid inhibitors; thyromimetics; tranquilizers; amyotrophic lateral sclerosis agent; cerebral ischemia agent; Paget's disease agent; unstable angina agent; vasoconstrictor; vasodilator; wound healing agent; xanthine oxidase inhibitor; and antineoplastic agents.

24. The method of claim 22, wherein the pharmaceutically active substance is Phenylephrine HCl.

25. The method of claim 20, wherein the active agent is a cosmetically active substance.

26. The method of claim 20, wherein the active agent is a nutritionally active substance

27. The method of claim 20, wherein the coating composition comprises a polyvinyl alcohol derived copolymer.

28. The method of claim 20, wherein the coating composition is applied to the substrate by spraying.

29. The method of claim 20, wherein the composition of matter is in the form chosen from the group consisting of tablet, caplet, pill, hard capsule, soft capsule, lozenge, powder, granule, suspension, and liquid.

30. The method of claim 20, wherein the composition of matter is in discrete dose units containing a predetermined amount of the pharmaceutically active agent.

31. A pharmaceutical composition produced according to the method of claim 20.

32. A pharmaceutical composition produced according to the method of claim 20 in the form of a tablet.
